(19) United States
(12) Patent Application Publication
(10) Pub. No.: US 2023/0145262 A1
(43) Pub. Date: May 11, 2023

(54) DEVICE WITH INSERT PLUG FOR OCCLUDING THE LEFT ATRIAL APPENDAGE

(71) Applicant: BOSTON SCIENTIFIC SCIMED, INC., MAPLE GROVE, MN (US)

(72) Inventors: Joshua Mark Inouye, Brooklyn Park, MN (US); Brian Joseph Tischler, Shoreview, MN (US); Harishankar Natesan, Shoreview, MN (US); Chen Jan-Hung, St. Paul, MN (US)

(73) Assignee: BOSTON SCIENTIFIC SCIMED, INC., MAPLE GROVE, MN (US)

(21) Appl. No.: 17/982,906

(22) Filed: Nov. 8, 2022

Related U.S. Application Data
(60) Provisional application No. 63/277,404, filed on Nov. 9, 2021.

(51) Int. Cl. A61B 17/12

(52) U.S. Cl. CPC A61B 17/12159; A61B 17/12122; A61B 17/12031; A61B 2017/1205

(57) ABSTRACT

An occlusion device for implantation in a left atrial appendage includes a proximal hub defining a recess configured to releasably connect with a delivery shaft, a frame connected to the proximal hub, a membrane coupled to and covering at least the proximal portion of the frame, the membrane comprising a material configured to block a passage of blood clots therethrough, and a plug configured to be received in the recess and remain in the recess after implantation.

### CROSS-REFERENCE TO RELATED APPLICATIONS

This application claims the benefit of priority of U.S. Provisional Application No. 63/277,404 filed Nov. 9, 2021, the entire disclosure of which is hereby incorporated by reference.

### TECHNICAL FIELD

The disclosure pertains to occlusion devices and more particularly to implantable occlusion devices for occluding the left atrial appendage of a patient, and methods for using such occlusion devices.

### BACKGROUND

Implanted occlusion devices are available for insertion into the left atrial appendage (LAA) of the heart. Such devices are used, for example, to block blood clots from passing out of the heart into the systemic circulation.

In general, these devices are delivered to the LAA through a catheter system that enters the venous circulation and approaches the left atrium through the atrial septum between the right and left side of the heart. The catheter is guided through the septum toward the ostium of the left atrial appendage. After acquisition and insertion into the LAA the implanted occlusion device is deployed, and fixed so that it remains in the appendage. Once positioned, the implanted occlusion device is released by the catheter, and the catheter system is removed. Over time, the exposed surface structures of the implanted occlusion device spanning the ostium of the LAA becomes covered with tissue. This process is called endothelization. Of the known occlusion devices and methods, each has certain advantages and disadvantages. There is an ongoing need to provide alternative occlusion devices as well as methods for using the occlusion devices.

### SUMMARY

This disclosure provides design, material, manufacturing method, and use alternatives for occlusion devices. An example occlusion device for implantation in a left atrial appendage includes a proximal hub defining a recess configured to releasably connect with a delivery shaft, a frame connected to the proximal hub and extending radially and then distally from the proximal hub, the frame having a proximal portion, an intermediate portion, and a distal portion, the frame comprising a plurality of struts extending between the proximal portion the distal portion, a membrane coupled to and covering at least the proximal portion of the frame, the membrane comprising a material configured to block a passage of blood clots therethrough, and a plug configured to be received in the recess and remain in the recess after implantation.

Alternatively or additionally to the embodiment above, the recess includes internal threads. Alternatively or additionally to any of the embodiments above, the plug includes external threads that mate with the internal threads on the recess.

Alternatively or additionally to any of the embodiments above, the recess has a first axial length and the plug has a second axial length, wherein the first axial length is greater than the second axial length.

Alternatively or additionally to any of the embodiments above, the plug has a connection element configured to releasably couple with a distal end of the delivery shaft.

Alternatively or additionally to any of the embodiments above, the plug is configured to move between a first position at a distal end of the recess during delivery, to a second position at a proximal end of the recess after the delivery shaft is disconnected.

Alternatively or additionally to any of the embodiments above, when the plug moves to the second position, the delivery shaft is automatically disconnected.

Alternatively or additionally to any of the embodiments above, the connection element is a cavity in a proximal face of the plug.

Alternatively or additionally to any of the embodiments above, the connection element is an off-center hole in a proximal face of the plug.

Alternatively or additionally to any of the embodiments above, the device further comprises a suture extending through the plug, through the distal end of the recess, the suture configured to extend through the delivery shaft.

Alternatively or additionally to any of the embodiments above, the plug includes an expandable material, and a distal region of the plug is fixed within a distal region of the recess.

Alternatively or additionally to any of the embodiments above, the plug includes a distal plate configured to engage a distal end of the proximal hub.

Alternatively or additionally to any of the embodiments above, the plug includes a plate and spring, with a first end of the spring fixed to the plate and a second end of the spring fixed to a distal end of the recess, wherein the spring is biased to extend the plate to a proximal end of the recess.

Another example occlusion device for implantation in a left atrial appendage includes a proximal hub defining a recess, a frame connected to the proximal hub and extending distally therefrom, the frame configured to move between a radially collapsed delivery configuration and a radially expanded configuration for engaging an interior wall of the left atrial appendage, a membrane coupled to and covering at least a proximal portion of the frame, the membrane comprising a material configured to block a passage of blood clots therethrough, and a plug disposed within the recess in the proximal hub and configured to releasably couple with a distal end of a delivery shaft, the plug configured to move between a first position at a distal end of the recess during delivery and a second position at a proximal end of the recess after the delivery shaft is disconnected, the plug configured to remain in the recess after removal of the delivery shaft.

An example system for implanting an occlusion device in a left atrial appendage includes a flexible delivery catheter. a delivery shaft extending through the delivery catheter, and an occlusion device comprising a proximal hub defining a recess configured to releasably connect with the delivery catheter, a frame connected to the proximal hub and extending radially and then distally from the proximal hub, the frame having a proximal portion, an intermediate portion, and a distal portion, the frame comprising a plurality of struts extending between the proximal portion the distal portion, a membrane coupled to and covering at least the proximal portion of the frame, the membrane comprising a material configured to block a passage of blood clots therethrough, a plug configured to be received in the recess and remain in the recess after implantation, the plug having a first connection element on a proximal end thereof, wherein the delivery shaft has a second connection element on a distal end thereof, the second connection element configured to releasably engage the first connection element on the plug.

Alternatively or additionally to the embodiment above, the distal end of the delivery shaft includes external threading, the recess includes internal threads, and the plug includes external threads, wherein when the second connection element of the delivery shaft engages the first connection element of the plug, the external threading on the plug and delivery catheter defines continuous threading that mates with the internal threading of the recess.

Alternatively or additionally to any of the embodiments above, the plug is configured to move between a first position at a distal end of the recess during delivery, to a second position at a proximal end of the recess after the delivery shaft is disconnected, wherein when the plug moves to the second position, the delivery shaft is automatically disconnected.

Alternatively or additionally to any of the embodiments above, one of the first and second connection elements is a cavity and the other of the first and second connection elements is a protrusion configured to engage the cavity.

Alternatively or additionally to any of the embodiments above, the first connection element is an off-center hole in a proximal face of the plug and the second connection element is an off-center protrusion extending distally from the distal end of the delivery shaft, the off-center hole configured to receive the off-center protrusion.

Alternatively or additionally to any of the embodiments above, the system further comprises a suture having two free ends, the suture extending through the plug, through the distal end of the recess, and through the delivery catheter, with the two free ends positioned proximal of a proximal end of the delivery catheter.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective proximal view of a prior art occlusion device;

FIG. 2 is a perspective proximal view of an example occlusion device according to the present disclosure;

FIG. 3 is a cross-sectional view taken along line 3-3 of FIG. 2;

FIG. 4 shows enlarged region 4 of FIG. 3;

FIG. 5A is a perspective view of an example delivery shaft and plug;

FIG. 5B is a close-up view of the distal end of the delivery shaft of FIG. 5A;

FIG. 5C is a close-up view of the plug of FIG. 5A;

FIGS. 6A-6C are side cross-sectional views of the delivery shaft and plug of FIG. 5A engaged with a portion of the occlusion device shown in FIG. 4, at various stages of deployment;

FIG. 7 is a perspective close-up view of the proximal hub of the occlusion device of FIG. 6C;

FIG. 8 is a perspective view of an alternative plug;

FIG. 9A is a side cross-sectional view of another example delivery shaft and plug;

FIG. 9B is a bottom view of the delivery shaft of FIG. 9A;

FIG. 9C is an enlarged view of the plug of FIG. 9A;

FIG. 10A is a side cross-sectional view of another example delivery shaft and plug;

FIG. 10B is a side cross-sectional view of the plug of FIG. 10A in an occlusion device during delivery; FIG. 11A is a side cross-sectional view of another example delivery shaft and plug coupled to an occlusion device during delivery;

FIG. 11B is a side cross-sectional view of the plug of FIG. 11A after removal of the delivery shaft;

FIG. 12 is a side cross-sectional view of an alternative plug disposed in an occlusion device;

FIG. 13A is a side cross-sectional view of another example delivery shaft and plug coupled to an occlusion device during delivery; and

FIG. 13B is a side cross-sectional view of the plug of FIG. 13A after removal of the delivery shaft.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently — such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

There is a continuing need to improve implanted occlusion devices as well as the methods and devices used to deliver them into the left atrial appendage (LAA). In some cases, exposed metal and threads on the proximal end of LAA occlusion devices exposed to the left atrium may become thrombogenic, leading to device-related thrombus (DRT). If DRT is present, patients cannot come off of Warfarin, defeating the purpose of the device. Furthermore, a thrombus formed on the proximal end of the device may break free, possibly causing an ischemic stroke. Reducing DRT is a desired advance in LAA closure devices.

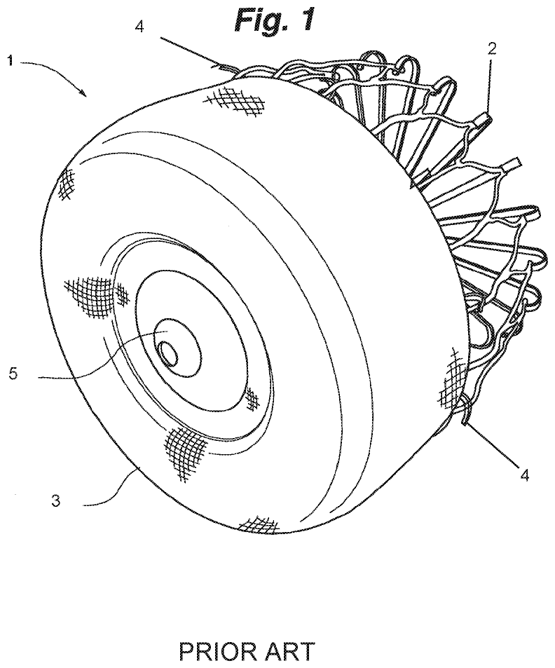

FIG. 1 illustrates an example implantable occlusion device 1, such as that disclosed in U.S. Pat. No. 9,913,652, which is incorporated herein by reference. The occlusion device 1 illustrated in FIG. 1 is shown in the expanded, deployed configuration. The occlusion device 1 may be delivered in a collapsed, compressed configuration through a catheter. It may be biased into the expanded or deployed configuration by the super-elastic nature of the material used to make the frame 2. In the deployed configuration, the device, and more specifically, the occlusion membrane 3 disposed over the proximal region of the device, spans the ostium of the LAA. The occlusion device 1 may include retention members associated with the periphery of the occlusion device 1. In some embodiments, the retention structure includes a series of barbs 4. The barbs 4 may be configured to penetrate tissue and retain the occlusion device 1 in the interior of the LAA during implantation. The occlusion device in FIG. 1 is seen from the proximal end in perspective. In situ, the occlusion membrane 3 completely covers the surface exposed to the interior of the heart chamber. An internally threaded connector 5 releasably couples the device to a threaded delivery shaft. In some cases, the exposed threading and recess of the connector 5 may contribute to DRT.

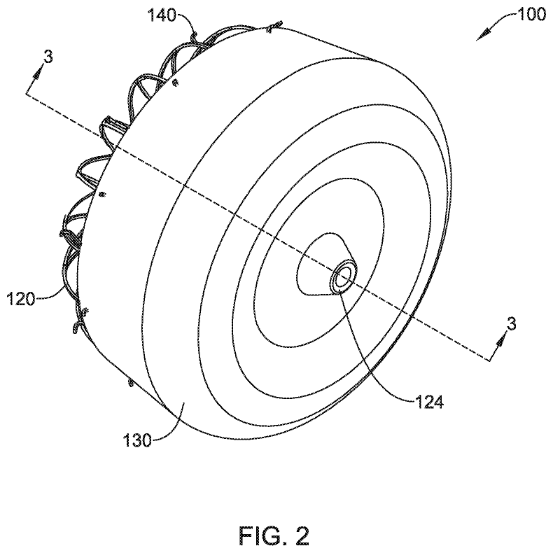

FIGS. 2-4 illustrate an occlusion device 100 with a plug 150 disposed in the recess within the proximal hub 124, flush with the proximal face of the device, thereby reducing thrombus risk. The structure of the frame 120 with barbs 140, and the occlusion membrane 130, as shown in FIG. 2, may be similar to the occlusion device 1 described in U.S. Pat. No. 9,913,652. The occlusion membrane 130 may cover the proximal end of the frame 120 and extend partially along sides of the frame 120. When the device is in position within an LAA, the occlusion membrane 130 spans the ostium and intercepts clots or mediates blood flowing in and out of the LAA. In some embodiments, the occlusion membrane 130 may be a filter member that blocks the passage of blood clots, but is permeable to blood flow there through. Alternatively, the occlusion membrane 130 may be of a material impermeable to blood flow. The occlusion membrane 130 may be fabricated from any suitable biocompatible materials. These materials include, but are not limited to, for example, ePTFE (e.g. Gore-Tex), polyester (e.g. Dacron®), PTFE (e.g. Teflon®), silicone, urethane, metal fibers, and other biocompatible polymers.

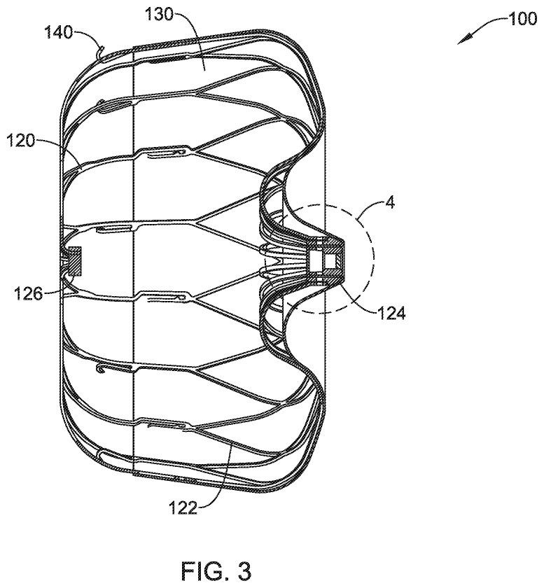

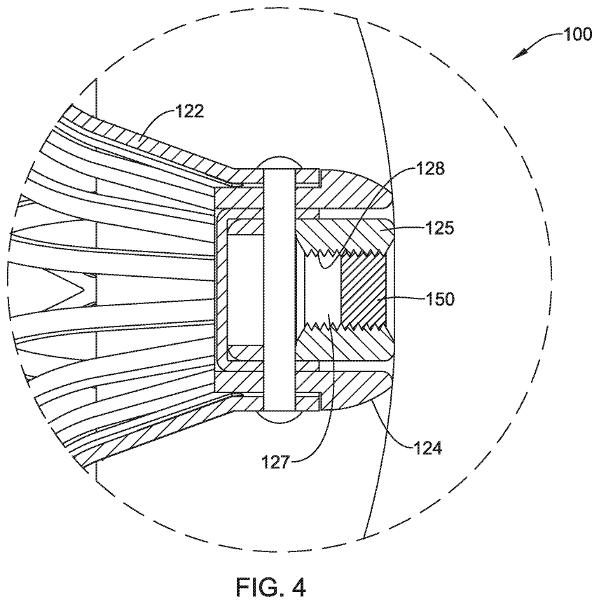

As shown in the cross-sectional view in FIG. 3, the occlusion device 100 may have a frame 120 extending radially and the distally from a proximal hub 124. The frame 120 may include a proximal portion, and intermediate portion, and a distal portion. The frame 120 may be formed from a plurality of struts 122 joined at the proximal hub 124 and a distal hub 126. A plurality of hooks or barbs 140 may extend radially outward from some of the struts 122. The barbs 140 may serve to secure the device in the LAA. As shown in the close-up view in FIG. 4, the plurality of struts 122 may be joined at a proximal hub 124. The proximal hub 124 may include an inner member 125 defining a recess 127 with internal threading 128. The internal threading 128 may extend along the entire axial length of the recess 127. The plug 150 is disposed within the recess 127.

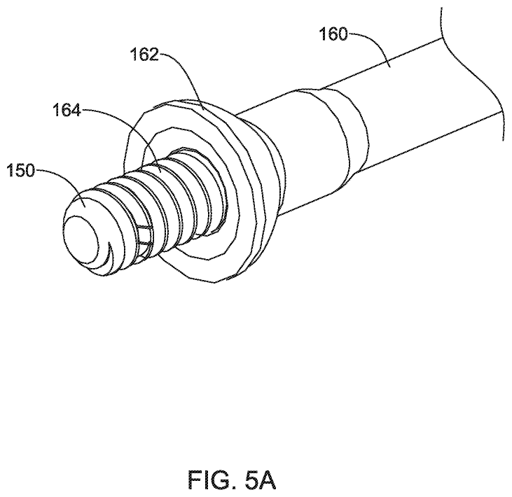

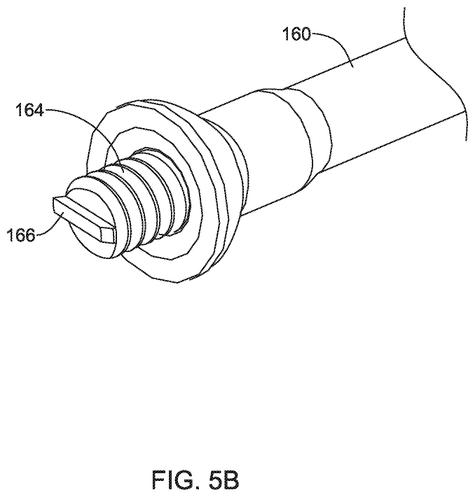

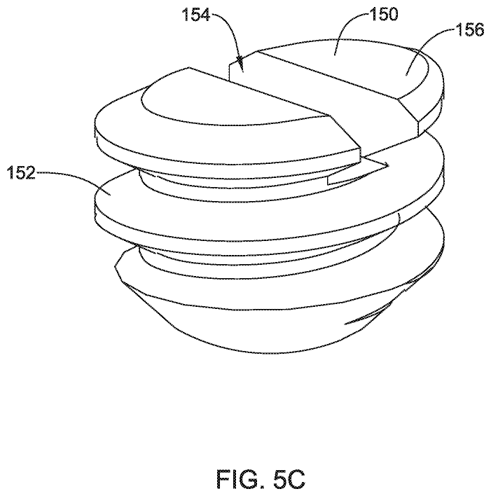

FIG. 5A illustrates a delivery shaft 160 configured to engage the occlusion device 100 during delivery through a catheter (not shown). The delivery shaft 160 may have a flared skirt 162 and a threaded distal end 164. A plug 150 may be releasably coupled with the threaded distal end 164. FIG. 5B is a close-up of the threaded distal end 164 of the delivery shaft 160, uncoupled from the plug 150. The plug 150 may have a first connection element configured to releasably couple with a second connection element on the distal end of the delivery shaft 160. In the example shown in FIG. 5B, the threaded distal end 164 may include a distally extending protrusion 166. FIG. 5C shows the plug 150 of FIG. 5A uncoupled from the delivery shaft 160. The proximal face 156 plug 150 may have a cavity 154 configured to receive the protrusion 166 in the threaded distal end 164 of the delivery shaft 160. the plug 150 may have external threading 152 configured to mate with the internal threading 128 in the recess of the proximal hub 124. The engagement between the protrusion 166 and cavity 154 may be similar to a screw driver and screw; tight enough to prevent the protrusion 166 from twisting out of the cavity 154 but loose enough to allow the protrusion 166 to be removed from the cavity 154 when the two parts are not threaded into the recess 127. In the embodiment shown in FIGS. 5A-5C, the threaded distal end 164 and the plug 150 have external threading that forms a continuous thread when the plug 150 is connected to the threaded distal end 164 of the delivery shaft 160. This continuous thread allows the plug 150 and threaded distal end 164 to be threaded into and out of the recess 127 as a single unit.

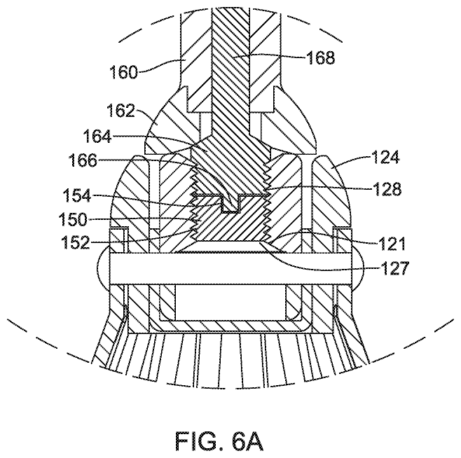

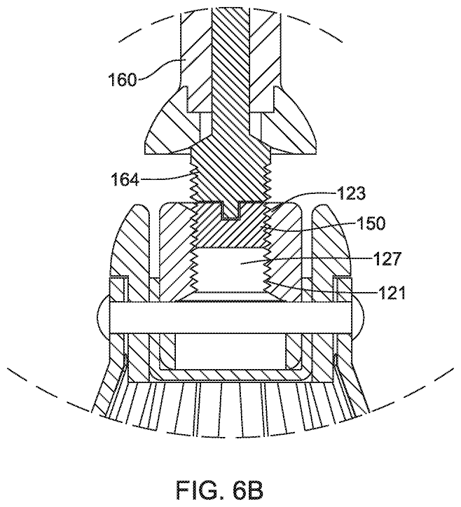

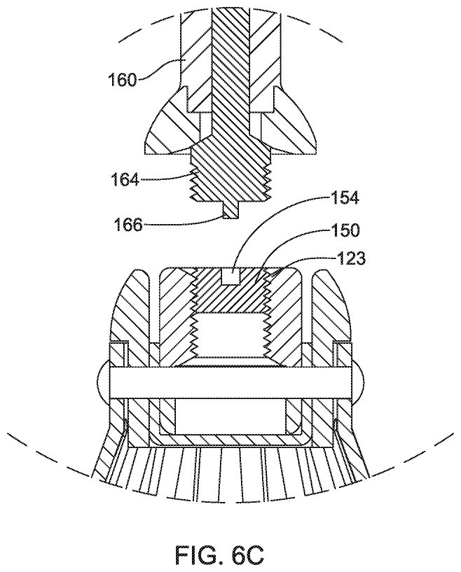

In FIG. 6A, the delivery shaft 160 and coupled plug 150 of FIG. 5A is shown threaded into the recess 127 within the proximal hub 124 of the occlusion device 100 of FIGS. 2-4. The flared skirt 162 may be configured to engage the proximal hub 124. The threaded distal end 164 may be provided on an inner wire 168 extending proximally through the delivery shaft 160. In some embodiments, the inner wire 168 may be slidable relative to the delivery shaft 160. In other embodiments, the threaded distal end 164 may be provided on the distal end of the delivery shaft 160. As illustrated, the threaded distal end 164 and external threading 152 on the plug 150 are threadingly engaged with the internal threading 128 of the recess 127 in the proximal hub 124.

The recess 127 may have a first axial length and the plug 150 may have a second axial length, with the first axial length being greater than the second axial length. This allows the plug 150 to move along the length of the recess 127 from a first position at the distal end 121 of the recess 127 during delivery, as shown in FIG. 6A, to a second position at the proximal end 123 of the recess 127 after the delivery shaft 160 has been disconnected, as shown in FIG. 6C. In some embodiments, the first axial length of the recess 127 may be at least 1.5 times the second axial length of the plug 150. In other embodiments, the first axial length may be twice the second axial length.

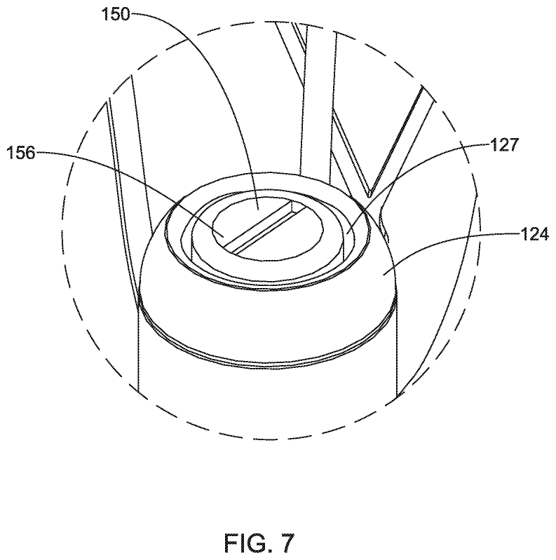

The protrusion 166 on the threaded distal end 164 of the delivery shaft 160 is disposed within the cavity 154 of the plug 150. This engagement causes the plug 150 to rotate with the delivery shaft 160, and to move proximally through the recess 127 as the delivery shaft 160 is rotated to withdraw the threaded distal end 164 proximally from the proximal hub 124. As shown in FIG. 6B, the delivery shaft 160 and threaded distal end 164 have been withdrawn proximally until the plug 150 is adjacent the proximal end 123 of the recess 127. As the plug 150 is coupled to the threaded distal end 164 of the delivery shaft 160 only by the protrusion 166 of the threaded distal end 164 sitting within the cavity 154 of the plug 150, once the threading on the threaded distal end 164 no longer engages the internal threading 128 in the recess 127, the protrusion 166 automatically slides out of the cavity 154, and the delivery shaft 160 is uncoupled from the plug 150. The plug 150 remains within the recess 127, flush with the proximal end 123 of the recess 127, as shown in FIG. 6C. The plug 150 covers the internal threading 128 and fills the recess 127, thereby reducing thrombus formation. FIG. 7 shows the plug 150 seated within the recess 127, with the proximal face 156 of the plug 150 flush with the proximal end of the proximal hub 124.

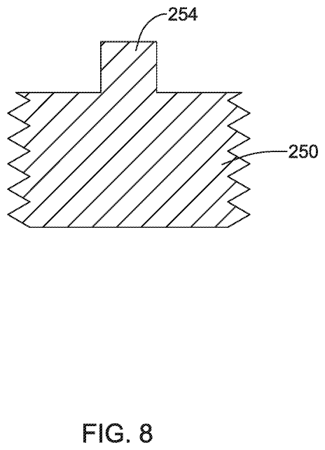

In another embodiment, the engagement structures of the plug 150 and the threaded distal end 164 of the delivery shaft 160 may be reversed. As shown in FIG. 8, the plug 250 may be externally threaded like the plug 150 described above, but may have a protrusion 254 which may be configured to engage a cavity in the distal end of the delivery shaft 160.

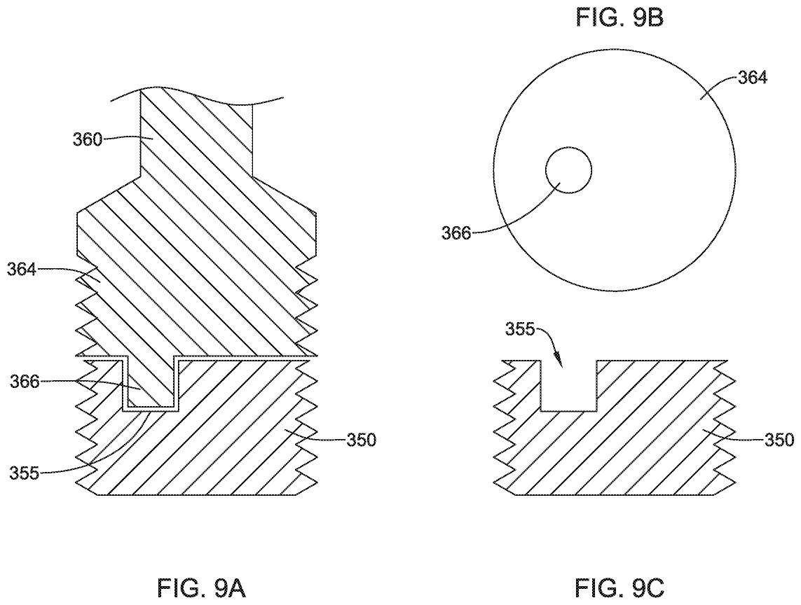

In another embodiment, instead of the cavity and protrusion coupling the plug 150 and delivery shaft 160 as discussed above, the connection element on the plug 350 may be an off-center axially extending hole 355 in the proximal face of the plug, configured to receive an off-center protrusion such as an elongated pin 366 extending distally and off-center from the threaded distal end 364 of the delivery shaft 360, as shown in FIG. 9A. FIG. 9B illustrates a distal end view of the threaded distal end 364 of the delivery shaft 360, showing the off-center position of the elongated pin 366. FIG. 9C illustrates a cross-sectional view of the plug 350 showing the off-center position of the axially extending hole 355 configured to receive the elongated pin 366. The off-center position of the hole 355 and elongated pin 366 provides the torque to rotate the plug 350 with the delivery shaft 360. The remaining structure of the plug 350 and delivery shaft 360 may be the same as the plug 150 and delivery shaft 160 described above. As with the above-described plugs 150, 150, the plug 350 covers the internal threading 128 and fills the recess 127 of the proximal hub on the occlusion device, thereby reducing thrombus formation.

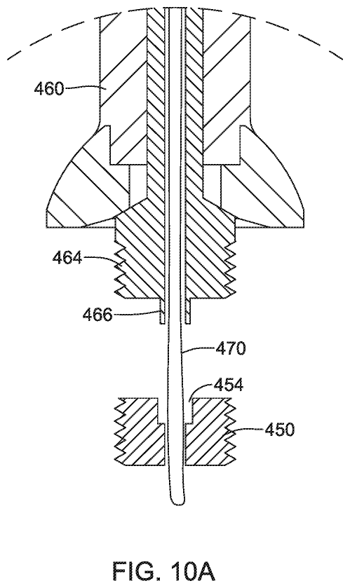

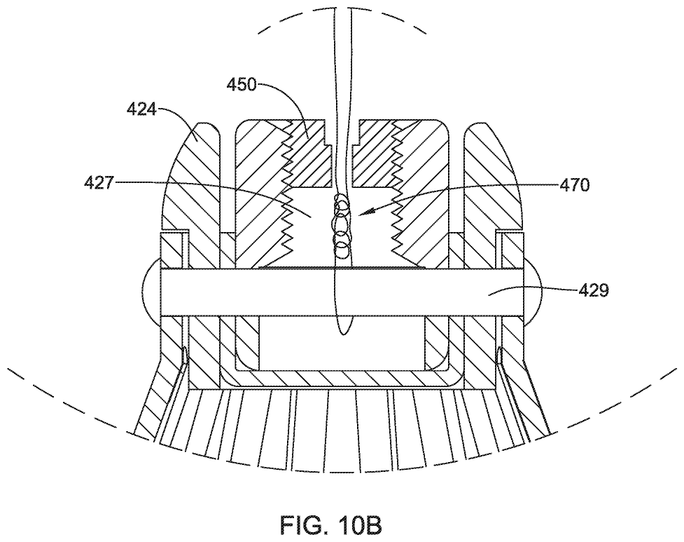

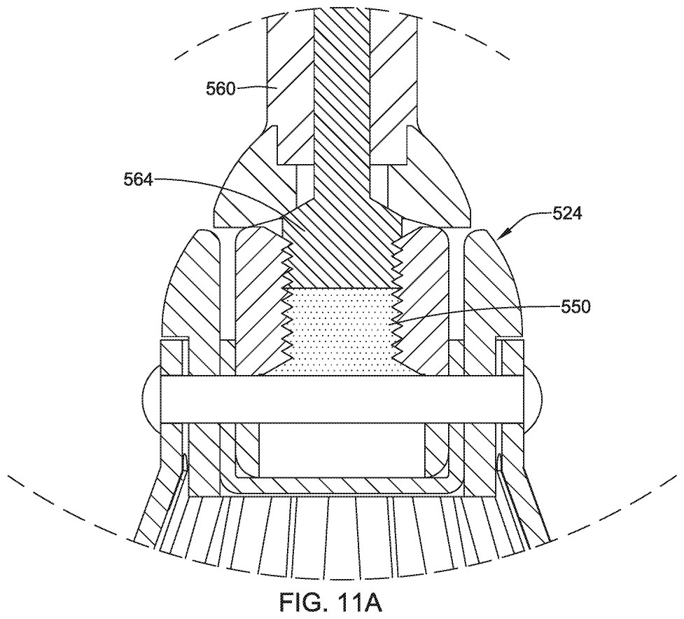

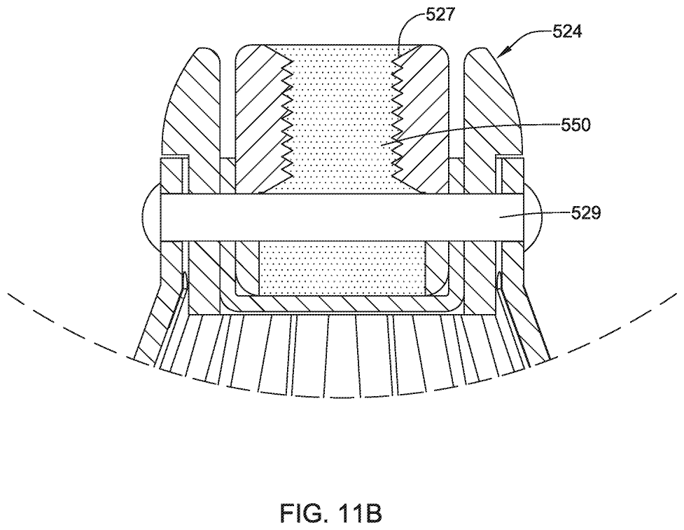

Similar to the embodiment shown in FIGS. 6A-6C, in addition to a protrusion 466 on the threaded distal end 464 of the delivery shaft 460 being disposed within the cavity 454 of the plug 450, the plug 450 may be coupled to the delivery shaft 460 by a suture 470, as shown in FIGS. 10A and 10B. FIG. 10A shows the suture 470 threaded through a lumen in the delivery shaft 460 and threaded distal end 464, through two separate holes in the plug 450 and back through the delivery shaft 460. The suture has two free ends and both free ends of the suture 470 may be disposed proximal of the delivery shaft 460. In some embodiments, the suture 470 may be threaded around a transverse pin 429 extending across the distal end of the recess 427 in the proximal hub 424, as shown in FIG. 10B. As the plug 450 is withdrawn to the proximal end of the recess 427, the rotation of the plug 450 may twist the suture 470 between the distal end of the plug 450 and the transverse pin 429, as shown in FIG. 10B. When the delivery shaft 460 rotates out of the recess 427 and releases the plug 450, the suture maintains a connection between the threaded distal end 464 of the delivery shaft 460 and the plug 450. If the position of the occlusion device needs to be adjusted, the suture 470 may help guide the protrusion 466 of the threaded distal end 464 of the delivery shaft 460 back in to the cavity 454 of the plug 450. The plug 450 may then be screwed back to the distal end of the recess 427 at which point the threaded distal end 464 of the delivery shaft 460 is also disposed within the recess 427 of the proximal hub 424. The position of the occlusion device can then be adjusted, followed by removal of the delivery shaft 460. Once the delivery shaft 460 is removed from the plug 450, one end of the suture 470 may be pulled, unthreading the suture from the delivery shaft 460 and the plug 450. As with the above-described plug 150, the plug 450 covers the threading and fills the recess 427 of the proximal hub on the occlusion device, thereby reducing thrombus formation.

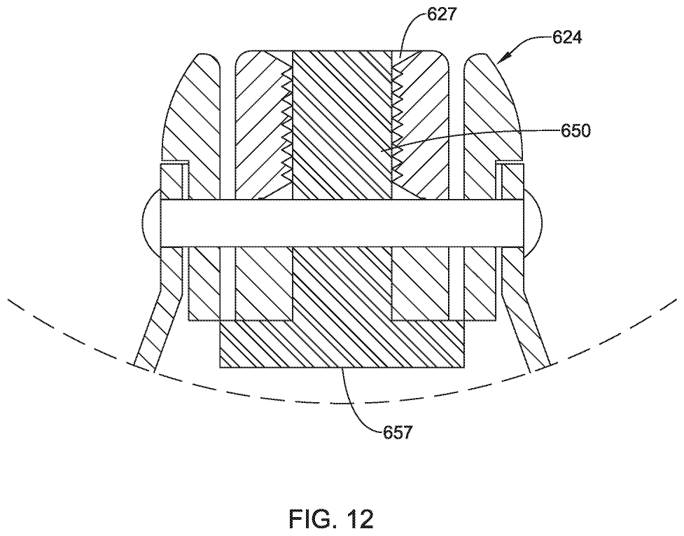

Instead of a threaded plug, an expandable plug 550 may be positioned within the proximal hub 524, compressed by the threaded distal end 564 of the delivery shaft 560, as shown in FIG. 11A. When the occlusion device is in the desired position, the delivery shaft 560 may be unscrewed from the proximal hub 524, allowing the expandable plug 550 to expand and fill the entire recess 527 in the proximal hub 524, as shown in FIG. 11B. The expandable plug 550 thereby reduces thrombus formation. The expandable plug 550 may be self-expandable in the absence of the compressive force of the threaded distal end 564 of the delivery shaft 560. For example, the expandable plug 550 may be made of expandable material such as foam, polymer, or silicone. In some examples, the expandable plug 550 may be fixed within the distal region of the recess 527 in the proximal hub 524 with at least one transverse pin 529 extending through opposite sidewalls of the proximal hub 524 and through the expandable plug 550, as shown in FIG. 11B. In some examples, the transverse pin 529 may include two separate pins extending into the expandable plug 550 from opposite sides through the proximal hub 524. In other embodiments, the distal end of the expandable plug 550 may be fixed to the distal end of the recess 527, such as with adhesive.

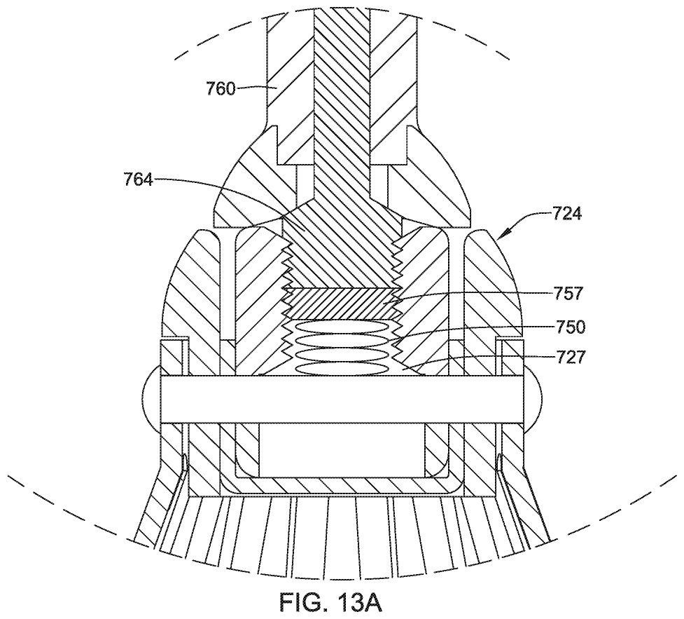

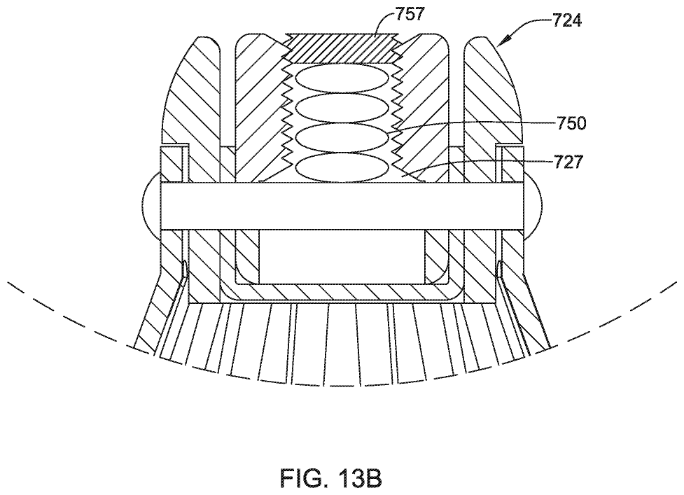

In a further embodiment, an expandable plug 650, similar to the expandable plug 550 described above, may include a distal plate 657 extending transverse across the recess 627 of the proximal hub 624 to engage a distal end of the proximal hub 624, as shown in FIG. 12. The distal plate 657 may prevent the expandable plug 650 from being removed proximally from the recess 627. The expandable plug 650 and the distal plate 657 may be a single monolithic piece, or the distal plate 657 may be made of a different material and fixed to the distal end of the expandable plug 650. As shown in FIG. 12, after removal of the delivery shaft, the expandable plug 650 has expanded to completely fill the recess in the proximal hub 624 of the occlusion device, thereby reducing thrombus formation. Alternatively, in another embodiment, the plug takes the form of a spring-loaded plate 757, as shown in FIGS. 13A and 13B. During delivery, the threaded distal end 764 of the delivery shaft 760 may be screwed into the internally threaded recess 727, abutting the plate 757 and compressing the spring 750. The spring 750 may have a first end fixed to the plate 757 and a second end fixed to the distal end of the recess 727 of the proximal hub 724. See FIG. 13A. The distal end of the threaded distal end 664 may be flat to engage a flat proximal surface of the plate 757. The spring 750 is biased such that as the threaded distal end 764 of the delivery shaft 760 is unscrewed and removed from the proximal hub 724 of the occlusion device, the spring 750 expands, extending the plate 757 to the proximal end of the recess 727, as shown in FIG. 13B. With the proximal surface of the plate 757 flush with the proximal end of the proximal hub 724, as shown in FIG. 13B, the plate 757 covers the recess 727 and its internal threading, thereby reducing thrombus formation.

While the protrusions 166, 254, 466 and their mating cavities 154, 454 are illustrated in the figures as being longitudinal protrusions and slots/grooves such as those seen on a conventional flat head screwdriver and screw, it will be understood that the geometry of the mating elements may be any that achieves the mating structure of the protrusion fitting within the cavity and allowing the two associated parts to rotate together and then separate as discussed above. Some example geometries for alternative protrusions and associated cavities include the following geometries of drill and driver bits: square, hex, pentagon, Phillips, Torx®, security Torx®, Tri-Wing®, spline, spanner, Pozidriv®, hex socket/Allen®, square recess, clutch, Mortorq®, Frearson, supadriv, Polydrive®, double square, Bristol, Torqset®, one-way, pentalobe, TP3, TTAP, and triangle/TA.

The plug 150, 250, 350, 450, 550, 650, 757 may be made from and/or coated with a polymer that resists thrombus, such as polytetrafluoroethylene (PTFE), or may be metallic, with or without a coating or surface texture that is advantageous for healing and biocompatibility. The coating may be an anti-thrombogenic coating. In some embodiments, the plug 150, 250, 350, 450, 550, 650, 757 may be made of or coated with a drug-eluting composition, for example an anti-thrombogenic drug composition.

To deploy the occlusion device 100 in an LAA, the occlusion device 100 is coupled to a delivery shaft 160, such as by the threaded distal end 164 to a plug 150 within a recess 127 in the proximal hub 124 of the occlusion device 100. The occlusion device may be radially collapsed within a flexible delivery catheter that is then percutaneously moved through the blood vessel to the desired location. When the end of the delivery catheter is adjacent the desired location, the device is deployed, allowing the frame 120 to radially expand to its relaxed size and shape configured for engaging an interior wall of the LAA, as depicted in FIG. 2. When the occlusion device 100 is properly positioned and fully deployed, it is untethered or released from the delivery shaft 160 by uncoupling the delivery shaft 160 from the plug 150, leaving the plug 150 flush with the proximal end of the proximal hub 124. The plug 150 fills the recess 127 in the proximal hub 124 and covers any internal threading in the recess, thereby reducing thrombus formation at the proximal hub 124. It will be understood that the above-described method of implanting an occlusion device applies equally to the other example plugs 250, 350, 450, 550, 650, 750.

The materials that can be used for the various components of the occlusion device 100, delivery shaft 160, and plug 150 (and/or other systems or components disclosed herein) and the various elements thereof disclosed herein may include those commonly associated with occlusion devices. For simplicity purposes, the following discussion refers to the occlusion device 100, delivery shaft 160, and plug 150 (and variations, systems or components disclosed herein). However, this is not intended to limit the devices and methods described herein, as the discussion may be applied to other elements, members, components, or devices disclosed herein.

In some embodiments, the occlusion device 100, delivery shaft 160, and plug 150 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the occlusion device 100, delivery shaft 160, and plug 150 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the occlusion device 100, delivery shaft 160, and plug 150 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the occlusion device 100, delivery shaft 160, and plug 150 (and variations, systems or components thereof disclosed herein) to achieve the same result.

In some embodiments, the occlusion device 100, delivery shaft 160, and plug 150 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the occlusion device 100 and plug 150 (and variations, systems or components thereof disclosed herein) may include and/or be treated with a suitable therapeutic agent. Some examples of suitable therapeutic agents may include anti-thrombogenic agents such as heparin, heparin derivatives, urokinase, and PPack (dextrophenylalanine proline arginine chloromethyl ketone).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An occlusion device for implantation in a left atrial appendage, the occlusion device comprising:
a proximal hub defining a recess configured to releasably connect with a delivery shaft;
a frame connected to the proximal hub and extending radially and then distally from the proximal hub, the frame having a proximal portion, an intermediate portion, and a distal portion, the frame comprising a plurality of struts extending between the proximal portion the distal portion;
a membrane coupled to and covering at least the proximal portion of the frame, the membrane comprising a material configured to block a passage of blood clots therethrough; and
a plug configured to be received in the recess and remain in the recess after implantation.

2. The occlusion device of claim 1, wherein the recess includes internal threads.

3. The occlusion device of claim 2, wherein the plug includes external threads that mate with the internal threads on the recess.

4. The occlusion device of claim 3, wherein the recess has a first axial length and the plug has a second axial length, wherein the first axial length is greater than the second axial length.

5. The occlusion device of claim 2, wherein the plug has a connection element configured to releasably couple with a distal end of the delivery shaft.

6. The occlusion device of claim 5, wherein the plug is configured to move between a first position at a distal end of the recess during delivery, to a second position at a proximal end of the recess after the delivery shaft is disconnected.

7. The occlusion device of claim 6, wherein when the plug moves to the second position, the delivery shaft is automatically disconnected.

8. The occlusion device of claim 7, wherein the connection element is a cavity in a proximal face of the plug.

9. The occlusion device of claim 7, wherein the connection element is an off-center hole in a proximal face of the plug.

10. The occlusion device of claim 7, further comprising a suture extending through the plug, through the distal end of the recess, the suture configured to extend through the delivery shaft.

11. The occlusion device of claim 1, wherein the plug includes an expandable material, and a distal region of the plug is fixed within a distal region of the recess.

12. The occlusion device of claim 11, wherein the plug includes a distal plate configured to engage a distal end of the proximal hub.

13. The occlusion device of claim 1, wherein the plug includes a plate and spring, with a first end of the spring fixed to the plate and a second end of the spring fixed to a distal end of the recess, wherein the spring is biased to extend the plate to a proximal end of the recess.

14. An occlusion device for implantation in a left atrial appendage, the occlusion device comprising:
a proximal hub defining a recess;
a frame connected to the proximal hub and extending distally therefrom, the frame configured to move between a radially collapsed delivery configuration and a radially expanded configuration for engaging an interior wall of the left atrial appendage;
a membrane coupled to and covering at least a proximal portion of the frame, the membrane comprising a material configured to block a passage of blood clots therethrough; and
a plug disposed within the recess in the proximal hub and configured to releasably couple with a distal end of a delivery shaft, the plug configured to move between a first position at a distal end of the recess during delivery and a second position at a proximal end of the recess after the delivery shaft is disconnected, the plug configured to remain in the recess after removal of the delivery shaft.

15. A system for implanting an occlusion device in a left atrial appendage, the system comprising:
a flexible delivery catheter;
a delivery shaft extending through the delivery catheter; and
an occlusion device comprising:
a proximal hub defining a recess configured to releasably connect with the delivery catheter;
a frame connected to the proximal hub and extending radially and then distally from the proximal hub, the frame having a proximal portion, an intermediate portion, and a distal portion, the frame comprising a plurality of struts extending between the proximal portion the distal portion;
a membrane coupled to and covering at least the proximal portion of the frame, the membrane comprising a material configured to block a passage of blood clots therethrough;
a plug configured to be received in the recess and remain in the recess after implantation, the plug having a first connection element on a proximal end thereof;
wherein the delivery shaft has a second connection element on a distal end thereof, the second connection element configured to releasably engage the first connection element on the plug.

16. The system of claim 15, wherein the distal end of the delivery shaft includes external threading, the recess includes internal threads, and the plug includes external threads, wherein when the second connection element of the delivery shaft engages the first connection element of the plug, the external threading on the plug and delivery catheter defines continuous threading that mates with the internal threading of the recess.

17. The system of claim 16, wherein the plug is configured to move between a first position at a distal end of the recess during delivery, to a second position at a proximal end of the recess after the delivery shaft is disconnected, wherein when the plug moves to the second position, the delivery shaft is automatically disconnected.

18. The system of claim 17, wherein one of the first and second connection elements is a cavity and the other of the first and second connection elements is a protrusion configured to engage the cavity.

19. The system of claim 17, wherein the first connection element is an off-center hole in a proximal face of the plug and the second connection element is an off-center protrusion extending distally from the distal end of the delivery shaft, the off-center hole configured to receive the off-center protrusion.

20. The system of claim 17, further comprising a suture having two free ends, the suture extending through the plug, through the distal end of the recess, and through the delivery catheter, with the two free ends positioned proximal of a proximal end of the delivery catheter.
